(19) United States
(12) Patent Application Publication
(10) Pub. No.: US 2006/0247790 A1
(43) Pub. Date: Nov. 2, 2006

(54) Shaped osteochondral grafts and methods of using same

(76) Inventor: William F. McKay, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/120,136

(22) Filed: Apr. 30, 2005

(51) Int. Cl. A61F 2/28

(52) U.S. Cl. 623/23.44; 623/23.63

(57) ABSTRACT

Described are plug grafts and in particular embodiments osteochondral plug grafts and grafting methods which utilize unique plug geometries and cooperative graft/host tissue interfaces to improve stability of grafted plugs within host tissue. Embodiments of the invention include harvested osteochondral or synthetic plug grafts having bore geometries other than circular cylinders and which are implantable in correspondingly prepared host sites to resist rotation and improve stability.

### BACKGROUND

The present invention relates generally to grafting for cartilage repair, and in one particular aspect to novel shaped osteochondral plug grafts and their use in articular cartilage resurfacing procedures.

As further background, lesions in articular cartilage, such as that which occurs in the knee joint, generally do not heal well due to the lack of nerves, blood vessels and a lymphatic system. Hyaline cartilage in particular has a limited capacity for repair, and lesions in this material without intervention typically form repair tissue lacking the biomechanical properties of normal cartilage.

A number of techniques are used to treat patients having damaged articular cartilage. Currently, the most widely used techniques involve non-grafting repairs or treatments such as lavage, arthroscopic debridement, and repair stimulation. Such repair stimulation is conducted by drilling, abrasion arthroplasty or microfracture. The goal is to penetrate into subchondral bone to induce bleeding and fibrin clot formation. This promotes initial repair. However, the resulting formed tissue is often fibrous in nature and lacks the durability of normal cartilage.

In a small number of procedures conducted today, cells grown in culture are transplanted into an articulating cartilage lesion. One such process involves the culture of a patient's own cells, and the reimplantation of those cells in defective cartilage. After implantation of the cells, an autologous periosteal flap with a cambium layer is used to seal the transplanted cells into place and act as mechanical barrier.

In another mode of treatment, osteochondral transplantation, also known as “mosaicplasty”, is used to repair articular cartilage. This procedures involves removing injured tissue from the articular defect and drilling cylindrical holes in the base of the defect and underlying bone. Cylindrical plugs of healthy cartilage and bone are obtained from another area of the patient, typically a lower-bearing region of the joint under repair, and are implanted into the drilled holes. In addition to the placement of autologous plugs of cartilage and underlying bone (osteochondral plugs), allograft osteochondral plugs have been suggested for use in repairing articular cartilage defects. Such allograft osteochondral plugs have been used clinically to some extent, in either fresh or frozen forms.

Despite work thus far in the area, needs remain for improved and/or alternative grafts and grafting techniques that are useful in the repair of articular cartilage. The present invention is addressed to these needs.

### SUMMARY

In one aspect, the present invention features the provision of plug implants having unique geometric and functional characteristics and their use in articular cartilage repair. Aspects of the present invention relate to osteochondral plug grafts including at least one bone portion exhibiting a cross-sectional profile other than that of a circle and configured for stable, durable and interlocking receipt within a corresponding surgically created opening in subchondral bone. Such osteochondral grafts or corresponding synthetic grafts or implants can be used in repair procedures in which the graft or other implant cooperates with the opening so as to provide a mechanical stop to resist rotation of the graft within the hole. Additionally or alternatively, such grafts/implants can cooperate with bone surfaces of adjacent implanted osteochondral plugs to provide such a mechanical stop to resist rotation. In this fashion, effective and stable graft materials and techniques are provided for the repair of patient articular cartilage.

One embodiment of the invention provides a method for repairing articular cartilage in a patient that includes implanting at least one osteochondral plug graft at an articular cartilage site in the patient, the plug graft including a cartilage layer attached to an underlying body of bone. As implanted, the body of bone includes a bone sidewall positioned adjacent a separate bone surface. The bone sidewall and adjacent bone surface together are configured to provide a mechanical interlock that resists rotation of the implanted osteochondral plug graft. The osteochondral plug graft can advantageously be an allograft osteochondral plug graft for implantation in a human. The mechanical stop can be provided by at least one region in which rotation of the plug graft would cause a wall or wall portion of the plug to impinge upon a wall of patient bone or a wall of an adjacent implanted plug and stop rotation of the plug. Thus, mechanical interlocks, apart from simple interference fits which involve only friction, are provided in accordance with this aspect of the invention. In other inventive aspects, synthetic plug grafts with corresponding features can be used in similar methods.

In another aspect, the present invention provides an osteochondral graft configured for stable implantation within a prepared surgical opening in subchondral bone of a patient at an articular cartilage site, the surgical opening having a three-dimensional contour other than a circular cylinder. The inventive osteochondral graft includes an osteochondral plug graft having a cartilage cap and a body of bone attached to the cartilage cap. The body of bone includes a stabilizing portion for receipt within the surgical opening, wherein the stabilizing portion of the bone body presents an external three-dimensional contour other than a circular cylinder. The stabilizing portion is further configured for mated receipt within the surgical opening to provide a mechanical interlock against rotation. In certain embodiments, osteochondral graft plugs include a body of bone having a cross-sectional profile that is non-circular but includes at least a portion defining an arc of a circle. Illustratively, such osteochondral graft plugs can take the form of multi-lobed grafts, wherein each lobe has a cross-sectional profile forming an arc of a circle. Such grafts may have two, three, four, or more such lobes. In further embodiments, osteochondral graft plugs of the invention can have bone bodies with polygonal cross-sectional profiles such as triangular, rectangular (including square), heptagonal, hexagonal, etc. cross-sectional profiles. Such grafts, or synthetic grafts having similar features, can for example be implanted into surgically prepared openings of similar shape to provide implanted grafts locked against rotation. As well, embodiments of the invention provide grafts including a bone body having an ovate cross-sectional profile, which can be implanted in openings of similar shape.

In a further aspect, the present invention provides a grafting system for treating an articular cartilage site comprising a first plug graft and a second plug graft. The first and second plug grafts are configured to cooperate with one another to nest, to mechanically lock at least one of the grafts against rotation, and/or to mechanically lock the grafts against lateral separation, when implanted at an articular cartilage site in a patient. The plug grafts can be osteochondral plug grafts, or synthetic plug grafts.

In another aspect, the present invention provides a graft for receipt within an opening in subchondral bone at an articular cartilage site of a patient, wherein the graft includes an osteochondral graft including a bone plug having an upper surface, sidewalls depending from the upper surface, and a lower surface, and a layer of cartilage attached to the upper surface of the bone plug. The bone plug further includes at least a portion wherein the bone plug sidewalls present a cross sectional profile selected from a non-circular profile that includes at least one circular arc, a polygonal profile, an ovate profile, and a multi-lobed profile having two to four lobes. Corresponding synthetic plug grafts also provide another feature of the invention.

In another embodiment the present invention provides a method for repairing an articular cartilage site in a patient that includes providing a prepared surgical opening in subchondral bone of a patient at an articular cartilage site and inserting a plurality of graft plugs into said surgical opening, wherein the plurality of plugs together provides a plug assembly substantially filling the opening. In some embodiments mated plug assemblies can be used to provide close packing of plug grafts with minimal gaps therebetween, and enhanced resurfacing effects at the site being treated. The graft plugs are desirably osteochondral plug grafts but in certain embodiments can also be synthetic plugs.

Another embodiment of the invention provides a graft system configured for stable implantation within a prepared surgical opening in subchondral bone of a patient at an articular cartilage site, wherein the system includes a plurality of graft plugs together providing a plug assembly configured to substantially fill the surgical opening.

Additional aspects as well as features and advantages of the invention will be apparent from the descriptions herein.

### BRIEF DESCRIPTION OF THE DRAWINGS

FIGS. 1 and 2 provide top and prospective views of a bilobal osteochondral graft of the invention respectively.

FIG. 3 shows a drill pattern for a hole for receiving a bilobal graft such as that depicted in FIGS. 1 and 2.

FIGS. 4 and 5 show top and prospective views of a trilobal osteochondral graft in accordance with the invention, respectively.

FIG. 6 shows a drill pattern for creating a hole for receiving a trilobal osteochondral graft such as that depicted in FIGS. 4 and 5.

FIGS. 7 and 8 provide top and prospective views of an osteochondral graft of the invention have four lobes, respectively.

FIG. 9 shows a drill pattern for creating a void for receiving an osteochondral graft such as that depicted in FIGS. 7 and 8.

FIGS. 10 and 11 depict perspective and top views of an oval-shaped osteochondral graft in accordance with the present invention.

FIGS. 12 and 13 provide top and perspective views of a generally square-shaped osteochondral graft in accordance with the invention.

FIGS. 13A and 13B provide top views of mating osteochondral graft assemblies of the invention.

FIGS. 14 and 15 depict osteochondral plug grafts of the invention which can be used in a nested arrangement.

FIG. 16 provides a top view of a nested arrangement of osteochondral grafts depicted in FIGS. 14 and 15.

FIGS. 17 through 19 depict top views of bilobal osteochondral grafts of the invention that can be used in a nested arrangement.

FIG. 20 provides a top view of an osteochondral graft of the invention having first and second lobes and a central region connecting the lobes.

FIG. 21 provides a top view of the graft of FIG. 20 in a nested arrangement with another similar graft.

FIGS. 22 and 23 provide top views of osteochondral plug grafts of the invention that can be used in a mated graft assembly.

FIG. 24 provides a top view of a mated graft assembly including the grafts depicted in FIGS. 22 and 23.

FIGS. 24 and 25 provide top and perspective views, respectively, of an osteochondral plug graft of the invention having a cruciform cross-sectional profile.

### DETAILED DESCRIPTION

For the purposes of promoting an understanding of the principles of the invention, reference will now be made to certain embodiments thereof and specific language will be used to describe the same. It will nevertheless be understood that no limitation of the scope of the invention is thereby intended, such alterations and further modifications in the described embodiments, and such further applications of the principles of the invention as illustrated therein being contemplated as would normally occur to one skilled in the art to which the invention relates.

As disclosed above, the present invention provides plug grafts such as osteochondral grafts having unique geometrical and functional characteristics as well as their use in novel grafting procedures. In particular aspects, plug grafts of the invention are arranged to provide and are used in a fashion wherein mechanical interlocking features resist rotation of the grafts when implanted, and/or wherein mechanical interlocking features resist lateral separation of adjacent implanted grafts, and/or wherein nested arrangements with adjacent plug grafts are achieved.

Osteochondral plug grafts of and for use in the invention can be harvested from the recipient or from a suitable human or other animal donor, from any appropriate structure including hyaline cartilage and underlying subchondral bone. Suitable harvest locations in large part occur in weight bearing joints of mammals, including humans. These harvest locations include, for example, articular cartilage and rib cartilage. A wide variety of articular cartilages may be used including for example those taken from articulating surfaces of the knee, hip, or shoulder joints. As specific examples, osteochondral plugs may be taken from the femoral condyle, the articulating surfaces of the knee, or the articulating surfaces of the shoulder.

Osteochondral grafts of the invention can be harvested at their final shape for implant or can be manipulated after harvest to provide the desired shape. In this regard, an osteochondral plug graft of the invention can have a cross sectional profile that is substantially constant or that varies along its length. For example, in certain embodiments, the cartilage layer or cap can have a cross sectional profile that is the same as the profile of the underlying bone plug, while in others the cartilage cap can have a cross sectional profile that differs from that of the bone plug. The latter may occur, for instance, in grafts having a cartilage cap that extends beyond the periphery of the bone plug, or terminates short of the periphery of the bone plug. As well, the bone plug itself may have a cross sectional profile that is constant along its length, or that varies along its length. Illustrative of the latter point, a cross sectional profile providing a unique, non-circular geometry as discuss herein may occur along only a portion of the bone plug, and yet provide stabilization features as described herein. These and other potential variations will be apparent to the skilled artisan from the descriptions herein.

In certain aspects of the present invention, an osteochondral plug graft for treating an articular cartilage defect includes a bone body with sidewalls having a cross-sectional profile other than a circular cylinder. In some inventive embodiments, such cross sectional profile will be that of a polygon, including equilateral and non-equilateral polygons, and regular and non-regular polygons. The polygon will typically having from three to about ten sides, including e.g. triangles, rectangles, pentagons, hexagons, cruciforms, etc. In other embodiments, such cross sectional profile will be non-circular, but will include at least one arc of a circle (sometimes herein referred to as a “circular arc”). These cross sections include desirable embodiments wherein the cross sectional profile of the bone body is defined by multiple, intersecting circular arcs, e.g. two, three, four or more intersecting circular arcs. In additional embodiments, the cross sectional profile presented by sidewalls of the bone body will be ovate, or will be multi-lobed, in some embodiments having from two to four lobes. Osteochondral plug grafts of the invention having such shapes can be configured for receipt within surgically prepared openings in a human or other mammalian knee, hip or shoulder joint to provide a mechanically interlocked arrangement as described herein, and to be capable of withstanding the biomechanical loads typically experienced at such joints without significant occurrence of fracture of the bone body of the osteochondral plug. Especially in embodiments in which protruding segments are provided to participate in mechanical locking (e.g. in multi-lobed devices), the cross-sectional profile and other physical attributes of the graft can be controlled to resist substantial fracture or break-off of the protruding segments under the ordinary loading conditions of a knee, hip, shoulder or other articular joint of a human or other mammalian patient in which the graft is to be implanted.

In the case of allograft osteochondral plugs, these can be either fresh (containing live cells) or processed and frozen or otherwise preserved to remove cells and other potentially antigenic substances while leaving behind a scaffold for patient tissue ingrowth. A variety of such processing techniques are known and can be used in accordance with the invention. For example, harvested osteochondral plugs can be soaked in an agent that facilitates removal of cell and proteoglycan components. One such solution that is known includes an aqueous preparation of hyaluronidase (type IV-s, 3 mg/ml), and trypsin (0.25% in monodibasic buffer 3 ml). The harvested osteochondral plugs can be soaked in this solution for several hours, for example 10 to 24 hours, desirably at an elevated temperature such as 37° C. Optionally, a mixing method such as sonication can be used during the soak. Additional processing steps can include decalcification, washing with water, and immersion in organic solvent solutions such as chloroform/methanol to remove cellular debris and sterilize. After such immersion the grafts can be rinsed thoroughly with water and then frozen and optionally lyophilized. These and other conventional tissue preservation techniques can be applied to the osteochondral grafts in accordance with the present invention.

Osteochondral grafts of the invention can be used in the repair of articular cartilage in patients, including for example that occurring in weight bearing joints such as those noted above and especially in the knee. The articular cartilage in need of repair can, for example, present a full thickness defect, including damage to both the cartilage and the underlying subchondral bone. Such defects can occur due to trauma or due to advanced stages of diseases, including arthritic diseases.

The articular cartilage site to be treated will typically be surgically prepared for receipt of the osteochondral graft. This preparation can include excision of patient cartilage and/or subchondral bone tissue at the site to create a hole or void in which the graft will be received. Tissue removal can be conducted in any suitable manner including for instance drilling and/or punching, typically in a direction substantially perpendicular to the articular cartilage layer at the site, to create a void having a depth approximating that of the graft to be implanted. In certain embodiments of the invention as discussed below, the opening for receiving the graft will be created using a drill or punch having a circular cross-section. Multiple, overlapping passes with the drill or punch are made, in order to create an opening having a cross-section defined by multiple, intersecting circular arcs. In this way, a multi-lobed surgical void can be created for receiving a correspondingly shaped osteochondral graft of the present invention in a mechanically locked condition. In other embodiments, a drill or punch that provides an opening with a non-circular cross-section with a single pass is used.

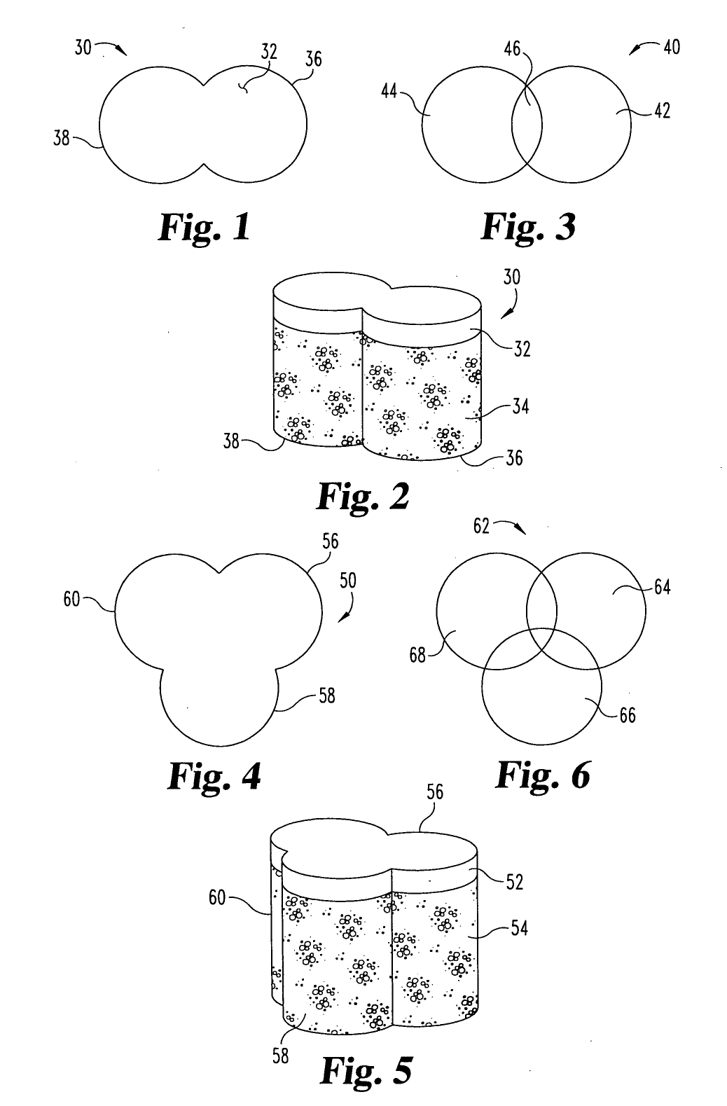

Turning now to a discussion of the Figures, shown in FIGS. 1 and 2 are top and perspective views, respectively, of a bilobal osteochondral graft product of the present invention. Graft 30 has an osteochondral structure including an underlying bone body 34 to which is attached a cartilage layer 32, desirably an articular (hyaline) cartilage layer. Bilobal graft 30 includes a first lobe 36 and a second lobe 38. Lobes 36 and 38 in the illustrated embodiment are provided as portions of right circular cylinders. Thus each lobe 36, 38 provides a cross-sectional profile that includes an arc of a circle. With reference now also to FIG. 3, which shows a drill pattern, the osteochondral graft 30 can thus be friction or interference fitted within an opening 40 in the patient tissue created by using a circular punch or drill to create holes 42 and 44 which overlap to an extent as shown at 46. In this manner, the osteochondral graft 30 may not only be frictionally fit into the opening 40, but this fit will also be of a nature that provides a mechanical interlock or stop against rotation of the graft 30 within the opening 40.

With reference now to FIGS. 4 and 5, shown are top and perspective views, respectively, of another multi-lobed osteochondral graft of the present invention. Graft 50 also includes a cartilage layer 52 attached to an underlying bone body 54. Graft 50 is provided having three lobes 56, 58, and 60. As in graft 30 discussed above, the lobes 56, 58, and 60 are provided as longitudinal portions of right circular cylinders and thus present external surfaces and a cross-section defined by multiple, intersecting circular arcs. With reference to FIG. 6, shown is a drill or punch pattern to create an opening 62 having a three-dimensional profile generally corresponding to that of the external surfaces of graft 50. In particular, a circular punch or drill can be used to create three overlapping cylindrical bores 64, 66, and 68, to define opening 62 presenting walls of a shape corresponding to the shape of graft 50. Again, in this manner, graft 50 can be fit within opening 62, optionally including a friction or interference fit, and will in cooperation with opening 62 provide a mechanical interlock to resist rotation of the graft 50 within the opening 62.

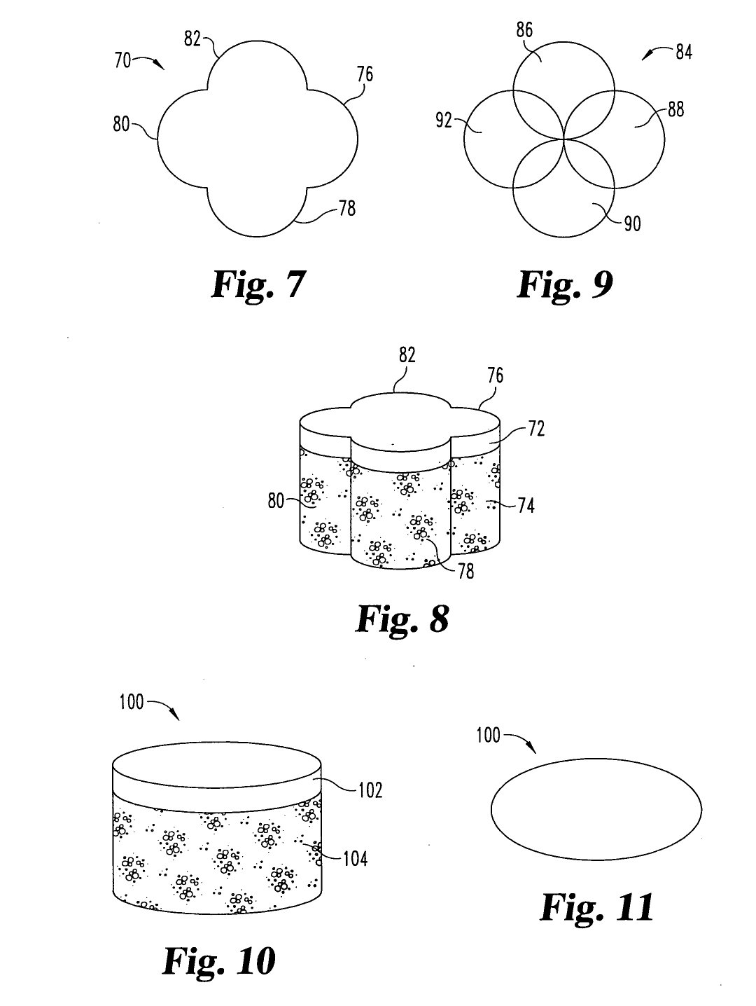

Shown in FIGS. 7 and 8 are top and perspective views of an osteochondral graft 70 of the present invention including four lobes. Graft 70 includes an overlying layer of cartilage 72 attached to an underlying bone body 74. Graft 70 includes four lobes 76, 78, 80, and 82. These lobes are provided as longitudinal sections or portions of right circular cylinders, and thus provide a cross-sectional profile defined by multiple interconnected circular arcs. In this manner, and with reference to FIG. 9, graft 70 can be implanted within an opening 84 of corresponding shape created with four overlapping passes of an instrument that forms right cylindrical bores 86, 88, 90, and 92 such as a circular punch, drill or other suitable mechanism. The graft 70 so implanted in opening 84 will be mechanically interlocked against rotation.

With reference now to FIGS. 10 and 11, shown are top and perspective views of one illustrative ovate osteochondral graft 100 of the present invention. Graft 100 thus includes a layer of cartilage 102 attached to an underlying body of bone 104. Graft 100 can be implanted into a correspondingly-dimensioned ovate opening created in the patient tissue at the implant site, and will thereby be mechanically locked against rotation within the implant site. Receipt of the graft 100 within the corresponding opening can also include an interference fit if desired. It will be understood that other symmetrical or unsymmetrical ovate shapes can also be used to provide similar functions.

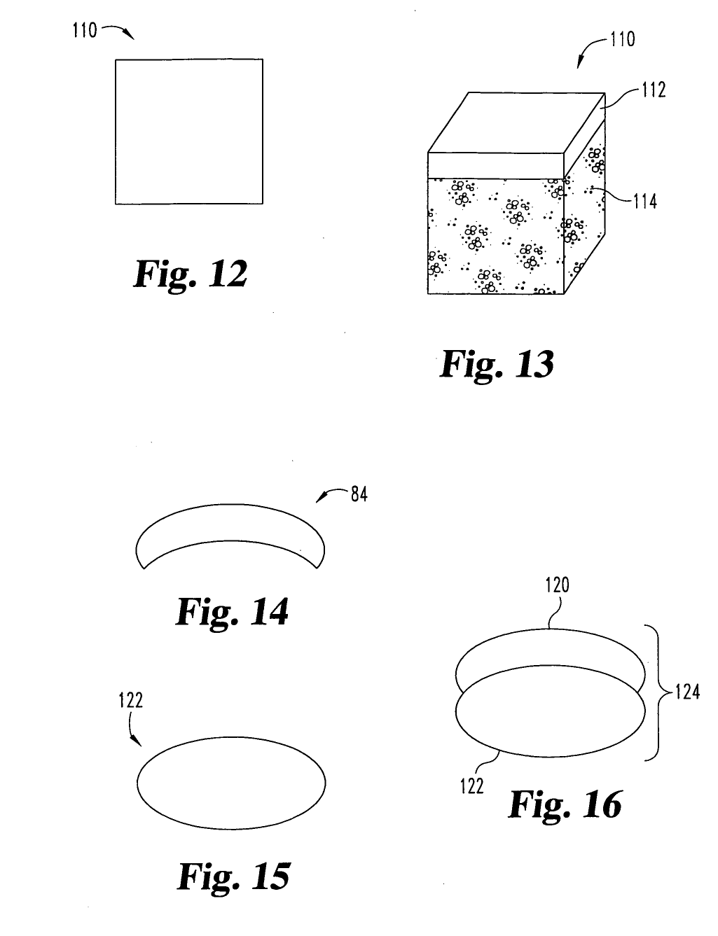

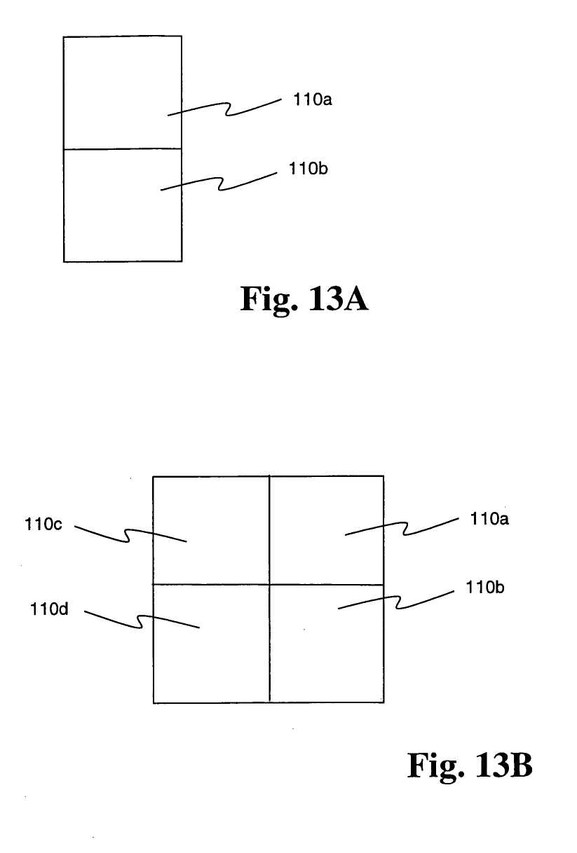

FIGS. 12 and 13 show a top and perspective view of a rectangular parallelepiped-shaped osteochondral graft of the present invention. Graft 110 includes a generally rectangular parallelepiped-shaped body of bone 114 attached to a generally rectangular parallelepiped-shaped layer of cartilage 112. Graft 110 can be implanted into a corresponding bore created at the implant site having a rectangular cross-section to achieve a non-rotating mechanical interlock fit within the opening. It will be understood that graft 110 depicts one possible parallelepiped-shape, in that other similar graft having differing rectangular cross-sectional shapes also form a part of present invention, including among others generally cube-shaped osteochondral grafts as well as those which are more or less elongate than that depicted for graft 110. In one illustrative example, a square or otherwise rectangular punch can be used to create a corresponding opening in the patient tissue for receiving graft 110 in a mated fit providing the non-rotatable arrangement.

A plurality of grafts 110 can be used to provide an advantageous graft assembly of the invention, configured for receipt within a single surgically prepared opening so as to mate with one another along one wall and substantially fill the opening. In this fashion, a close-fit between adjacent plugs can be achieved, providing better filling of the articular defect under treatment. Specifically with reference to FIG. 13A, shown are two graft plugs 110a and 110b mated together along one wall and received within the same, rectangular-shaped surgical opening. In certain embodiments, such graft assemblies include more than two graft plugs, for example from two to six plugs. In this regard, depicted in FIG. 13B is a graft assembly including four rectangular (square) graft plugs 110a, 110b, 110c and 110d mated together and closely packed within a single surgical opening in patient subchondral bone. It will be understood that graft plugs having cross sectional profiles other than rectangles can also be used in mated fashion in a surgical opening, wherein walls of the plugs are configured to conform to one another along an extended length when the plugs are received together within the opening. Such mating or conforming walls can have generally straight or curved profiles or combinations thereof, or any other suitable mating shape.

FIGS. 14 and 15 provide top views of osteochondral graft 120 and 122 of the invention which are configured to nest with one another as implanted. Specifically, shown in FIG. 16 is a nested assembly 124 including graft 120 and graft 122, wherein at least one arcuate convexity or protrusion of one of the grafts (e.g. graft 122) is matingly received within a generally corresponding arcuate concavity or cut-out region of the adjacent graft (graft 120). Such nested arrangements can be used to provide advantageous close packing of multiple implanted osteochondral grafts to facilitate an effective fill of a larger damaged tissue region, and/or can participate in preventing rotation of one or more of the implanted, nested grafts.

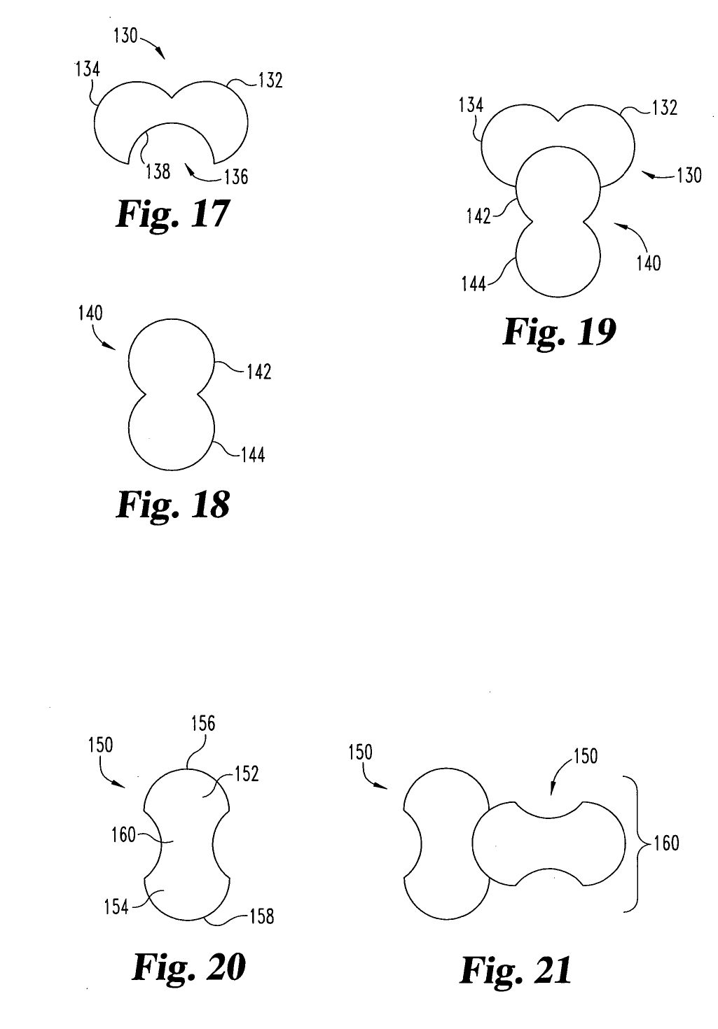

FIGS. 17 and 18 illustrate another set of osteochondral grafts which are nestable with one another. In particular, graft 130 presents two lobes 132 and 134 and a concave cut-out 136 presenting a generally concave surface 138. Osteochondral graft 140 is similar to that depicted in FIGS. 1 and 2 and thus presents a first lobe 142 and a second lobe 144. In the illustrated grafts 130 and 140, each graft presents external surfaces that correspond to longitudinal sections of right circular cylinders, as does their nested overall profile. In this regard, referring to FIG. 19, graft 140 is shown partially nested within graft 130, with a portion of lobe 142 of graft 140 received within concavity 136 and abutted against concave surface 138. The nested configuration shown in FIG. 19 can be inserted into a corresponding unitary opening created in the patient tissue using a series of right cylindrical bores made in an overlapping pattern. The assembled graft shown in FIG. 19 both nests and provides a mechanical interlock against rotation when implanted.

With reference to FIG. 20, shown is another osteochondral graft 150 in accordance with the present invention. Graft 150 includes a first lobe 152 and a second lobe 154 presenting exterior surfaces 156 and 158 consistent with those of circular cylinders. Lobes 152 and 154 are interconnected by a central portion 160 which presents a generally concave surface on each side. In this manner, as depicted in FIG. 21, a number of grafts 150 can be nested together as implanted to provide a nested assembly 160. An opening of corresponding shape can be created in the patient tissue to receive the nested assembly 160 using a punch having a shape corresponding to the exterior shape of graft 150, or using a drill or other device manipulable to create an opening of the appropriate size and shape.

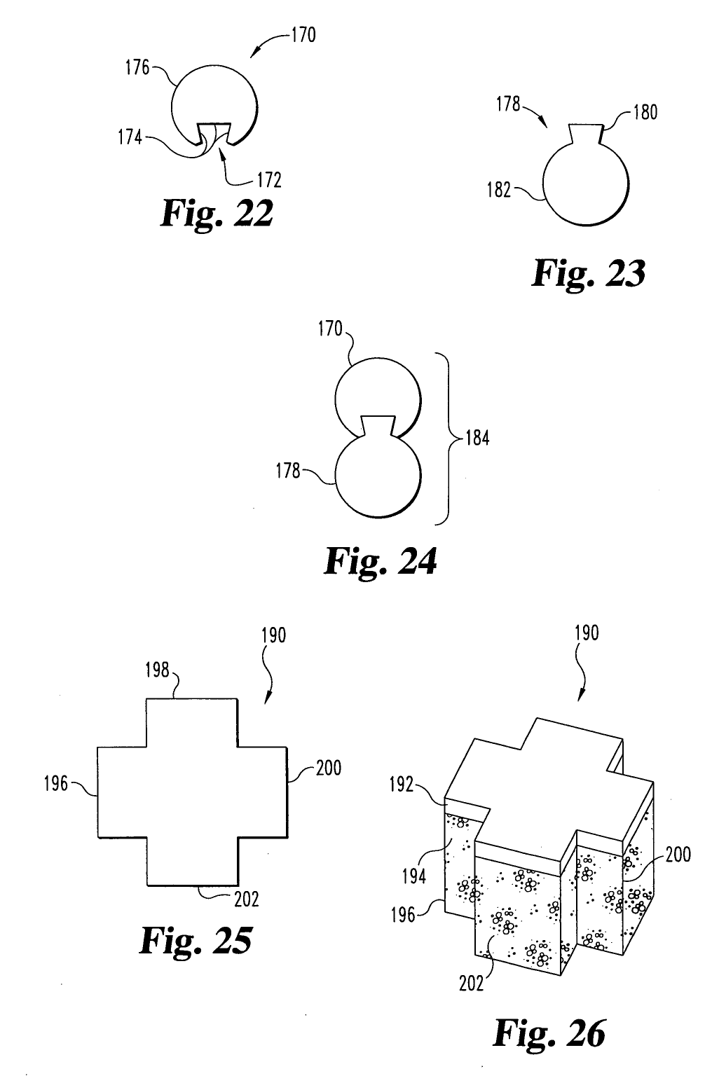

FIGS. 22-24 illustrate additional osteochondral grafts and assemblies of the invention, which are configured to mechanically interlock with one another to resist lateral separation, e.g. by providing a interleaved joint (e.g. as provided in a dovetail or other similar undercut arrangement) between portions of the grafts. Specifically as shown, osteochondral graft 170 is provided generally as right circular cylinder having a dovetail cut-out 172 extending longitudinally therein. Dovetail cut-out 172 presents a series of inner walls 174 for receiving and mechanically restraining a corresponding dovetail protrusion. Graft 170 presents an arcuate outer wall 176 consistent with that of a circular cylinder, for receipt within a corresponding cylinder bore at the implant site. Osteochondral graft 178 (FIG. 23) includes a generally circular cylindrical portion 182 and a dovetail-shaped protrusion 180 extending along its length. In this manner, as shown in FIG. 24, grafts 170 and 178 can be implanted together in an interleaved fashion forming a dovetail joint between the two, whereby they are mechanically together against lateral separation. As well, the interleaved assembly 184 can be implanted within an opening in the patient tissue of a corresponding shape, which in turn can be created as overlapping circular bores using conventional drills, punches or other equipment for creating the same. It will be understood that the assembly 184 depicts one illustrative embodiment of interleaved, mechanically locked grafts, and that many other arrangements which provide for interleaving portions of adjacent grafts so as to provide locking or resistance to lateral separation and/or rotation can be used within the spirit and scope of the present invention.

FIGS. 25 and 26 provide top and perspective views, respectively, of a cruciform osteochondral plug graft 190 of the present invention. Graft 190 includes a cartilage layer 192 attached to an underlying bone body 194, and have a cross sectional profile defined by four generally rectangular projecting segments 196, 198, 200 and 202, forming an overall cruciform or cross-shaped profile. Graft 190 can be implanted into a corresponding bore created at the implant site having a cruciform cross-section to achieve a non-rotating mechanical interlock fit within the opening. Such an opening can be surgically prepared for example using a correspondingly-shaped punch, or using multiple overlapping passes of an appropriately rectangular punch.

While certain discussions above have focused upon the use of harvested osteochondral plug grafts, in other aspects of the invention, plug grafts of and for use in the invention can be manufactured from other materials or components. Illustratively, plug grafts adapted for receipt in surgical openings in subchondral bone at articular sites, and desirably for integration with the subchondral bone, can be synthesized from natural or synthetic materials. For example, plug bodies can be synthesized from biopolymers or from synthetic polymers (bioabsorbable and non-bioabsorbable synthetic polymers), ceramics, or combinations thereof. Illustrative synthetic bioabsorbable, biocompatible polymers, which may act as suitable matrices for plug bodies can include poly-alpha-hydroxy acids (e.g. polylactides, polycaprolactones, polyglycolides and their copolymers, such as lactic acid/glycolic acid copolymers and lactic acid/caprolactone copolymers), polyanhydrides, polyorthoesters, polydioxanone, segmented block copolymers of polyethylene glycol and polybutylene terephtalate (Polyactivea3, poly (trimethylenecarbonate) copolymers, tyrosine derivative polymers, such as tyrosine-derived polycarbonates, or poly (ester-amides). Suitable ceramic materials include, for example, calcium phosphate ceramics such as tricalcium phosphate, hydroxyapatite, and biphasic calcium phosphate. These or other suitable materials can be used to form plug grafts useful in articular cartilage resurfacing procedures. In this regard, such grafts may have a uniform composition throughout, or may vary, for instance having a plug body formed of a first, relatively strong and loadbearing material (e.g. a ceramic, polymer or composite), and a cap formed of another material to provide the articulating surface formed by another material, for example a relatively smooth polymer layer. These and other variants will be apparent to the skilled artisan from the descriptions herein.

Plug grafts of the invention can be used in conjunction with other materials helpful to the treatment. For example, the grafts can be used in combination with a growth factor, and especially a growth factor that is effective in inducing formation of bone and/or cartilage tissue. Desirably, the growth factor will be from a class of proteins known generally as bone morphogenic proteins (BMPs), and can in certain embodiments be recombinant human (rh) BMPs. These BMP proteins, which are known to have osteogenic, chondrogenic and other growth and differentiation activities, include rhBMP-2, rhBMP-3, rhBMP4 (also referred to as rhBMP-2B), rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-11, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in U.S. Pat. Nos. 5,965,403 and 6,331,612. Other compositions which may also be useful include Vgr-2, and any of the growth and differentiation factors [GDFs], including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The disclosures of all of these patents and applications are hereby incorporated herein by reference. Also useful in the present invention are heterodimers of the above and modified proteins or partial deletion products thereof. These proteins can be used individually or in mixtures of two or more. rhBMP-2 is preferred.

The BMP may be recombinantly produced, or purified from a protein composition. The BMP may be homodimeric, or may be heterodimeric with other BMPs (e.g., a heterodimer composed of one monomer each of BMP-2 and BMP-6) or with other members of the TGF-beta superfamily, such as activins, inhibins and TGF-beta 1 (e.g., a heterodimer composed of one monomer each of a BMP and a related member of the TGF-beta superfamily). Examples of such heterodimeric proteins are described for example in Published PCT Patent Application WO 93/09229, the specification of which is hereby incorporated herein by reference. The amount of osteogenic protein useful herein is that amount effective to stimulate increased osteogenic activity of infiltrating progenitor cells, and will depend upon several factors including the size and nature of the defect being treated, and the carrier and particular protein being employed. In certain embodiments, the amount of osteogenic protein to be delivered will be in a range of from about 0.05 to about 1.5 mg.

An osteogenic protein used to form bone can also be administered together with an effective amount of a protein which is able to induce the formation of tendon- or ligament-like tissue in the implant environment. Such proteins include BMP-12, BMP-13, and other members of the BMP-12 subfamily, as well as MP52. These proteins and their use for regeneration of tendon and ligament-like tissue are disclosed for example in U.S. Pat. Nos. 5,658,882, 6,187,742, 6,284,872 and 6,719,968 the disclosures of which are hereby incorporated herein by reference.

Growth factor may be applied to the tissue source in the form of a buffered aqueous solution. Other materials which may be suitable for use in application of the growth factors in the methods and products of the present invention include carrier materials such as collagen, milled cartilage, hyaluronic acid, polyglyconate, degradable synthetic polymers, demineralized bone, minerals and ceramics, such as calcium phosphates, hydroxyapatite, etc., as well as combinations of these and potentially other materials.

Other biologically active materials may also be used in conjunction with osteochondral grafts of the present invention. These include for example cells such as human allogenic or autologous chondrocytes, human allogenic cells, human allogenic or autologous bone marrow cells, human allogenic or autologous stem cells, demineralized bone matrix, insulin, insulin-like growth factor-1, interleukin-1 receptor antagonist, hepatocyte growth factor, platelet-derived growth factor, and Indian hedgehog and parathyroid hormone-related peptide, to name a few.

In certain modes of practice, suitable organic glue material can be used to help secure the graft in place in the implant area. Suitable organic glue material can be obtained commercially, such as for example; TISSEEL® or TISSUCOL® (fibrin based adhesive; Immuno AG, Austria), Adhesive Protein (Sigma Chemical, USA), Dow Corning Medical Adhesive B (Dow Corning, USA), fibrinogen thrombin, elastin, collagen, casein, albumin, keratin and the like.

When used, the growth factor and/or other material(s) can be applied directly to the plug graft and/or to the site in need of repair. For example, the growth factor and/or other material may be physically applied to the graft (e.g. the bone and/or cartilage tissue of an osteochondral graft) through spraying or dipping, or using a brush or other suitable applicator, such as a syringe. Alternatively, or in addition, amounts of the growth factor or other material(s) can be directly applied to the site in need of tissue repair, for example by filling or coating the surgically-prepared opening with one or more of these substances.

Instability of grafted plugs within prepared defect sites can contribute to delayed or failed incorporation of the grafted material with the patient tissue. Osteochondral plug grafts and grafting methods of the present invention can be used in certain aspects of the invention to provide improved implant stabilization, more rapid or complete incorporation of the graft into patient tissue, and/or an enhanced ability to restore articular cartilage defects. In addition, the use of circular cross section graft plugs in adjacent, separate surgical openings leaves gaps between grafts, which can present a relatively non-uniform articulating surface and can also provide pathways for the migration of synovial fluids into the subchondral bone, which may impair graft integration or otherwise deleteriously affect patient outcome. In certain aspects of the invention, grafts having non-circular cross section can be used adjacent to one another, including in the same surgical opening, in a fashion that leaves fewer or smaller gaps in the resurfaced area and enhances the grafting procedure. In these regards, it will be understood that while these particular enhanced features can be provided in certain inventive aspects, they are not required in all embodiments or broader features of the present invention. It should also be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. All publications, patents and patent applications cited in this specification are herein incorporated by reference as if each individual publication, patent or patent application was specifically and individually indicated to be incorporated by reference as set forth in its entirety herein.

1. A method for repairing articular cartilage in a patient, comprising:
implanting at least one osteochondral plug graft at an articular cartilage site in the patient;
said plug graft comprising a cartilage layer attached to an underlying body of bone;
said body of bone, as implanted, comprising a bone sidewall positioned adjacent to a bone surface;
said bone sidewall and said bone surface together configured to provide a mechanical interlock to resist rotation of the implanted osteochondral plug graft.

2. The method of claim 1, wherein the bone surface is a surface of an adjacent osteochondral plug graft.

3. The method of claim 1, wherein the bone surface is a surface of bone of the patient.

4. The method of claim 1, wherein the body of bone has at least a portion having a polygonal cross-section.

5. The method of claim 4, wherein the articular cartilage site includes a hole having a polygonal cross-section corresponding to that of the body of bone, and wherein the body of bone is received in said hole.

6. The method of claim 5, wherein the polygonal cross-section is a rectangle.

7. The method of claim 6, wherein the polygonal cross section is a square.

8. The method of claim 1, wherein the body of bone has at least a portion having a multi-lobed cross section.

9. (canceled)

10. (canceled)

11. (canceled)

12. The method of claim 8, wherein the cross section includes from 2 to 6 circular arcs.

13. The method of claim 12, also comprising preparing the articular cartilage site for receipt of the osteochondral graft by creating a void in subchondral bone of the site, the void having a periphery defined by multiple overlapping right circular cylinders.

14. (canceled)

15. (canceled)

16. The method of claim 1, wherein the body of bone includes at least a portion having an oval-shaped cross section.

17. The method of claim 16, wherein the articular cartilage site is prepared to have an oval-shaped opening corresponding to the oval-shaped cross section of the body of bone, and wherein the at least a portion of the body of bone having an oval-shaped cross section is received in said oval-shaped opening.

18. The method of claim 1, wherein said sidewall and said bone surface further provide an interference fit against one another.

19. (canceled)

20. (canceled)

21. (canceled)

22. (canceled)

23. The method of claim 1, also comprising administering an osteogenic protein to the articular cartilage site.

24. The method of claim 23, wherein the osteogenic protein is a bone morphogenic protein (BMP).

25. The method of claim 24, wherein the BMP comprises recombinant human BMP-2.

26. (canceled)

27. (canceled)

28. A method for repairing an articular cartilage site in a patient, comprising:
implanting at least one plug body at an articular cartilage site in the patient;
said plug body, as implanted, comprising a sidewall positioned adjacent to a contact surface, said contact surface provided by subchondral bone of the patient and/or a surface of an adjacent plug body;
said sidewall and contact surface together configured to provide a mechanical interlock to resist rotation of the implanted plug body.

29. An osteochondral graft configured for stable implantation within a prepared surgical opening in subchondral bone of a patient at an articular cartilage site, the surgical opening having a three-dimensional contour other than a circular cylinder, the osteochondral graft comprising:
an osteochondral graft plug having a cartilage cap and a body of bone attached to the cartilage cap;
said body of bone including a stabilizing portion for receipt within the surgical opening;
said stabilizing portion of said body of bone presenting a three-dimensional contour other than a circular cylinder and configured for mated receipt within the surgical opening to provide a mechanical interlock against rotation.

30. The osteochondral graft of claim 29, wherein the surgical opening and the stabilizing portion have corresponding polygonal cross sections.

31. The osteochondral graft of claim 30, wherein the polygonal cross-sections are rectangular.

32. (canceled)

33. The osteochondral graft of claim 29, wherein the surgical opening and the stabilizing portion have corresponding multi-lobed cross sections.

34. The osteochondral graft of claim 33, wherein the cross sections are defined by multiple intersecting circular arcs.

35. (canceled)

36. (canceled)

37. (canceled)

38. (canceled)

39. The osteochondral graft of claim 29, wherein the surgical opening and stabilizing portion have corresponding ovate cross sections.

40. The osteochondral graft of claim 29, wherein said stabilizing portion is further sized and configured to provide an interference fit when received in said surgical opening.

41. The osteochondral graft of claim 29 wherein the patient is a human, and wherein the osteochondral graft is effectively configured to withstand biomechanical loads experienced by a human knee.

42. (canceled)

43. (canceled)

44. (canceled)

45. The osteochondral graft of claim 29, also comprising an osteogenic protein carried by said body of bone.

46. The osteochondral graft of claim 45, wherein the osteogenic protein is a bone morphogenic protein (BMP).

47. The osteochondral graft of claim 46, wherein the BMP comprises recombinant human BMP-2.

48. A medical implant configured for stable implantation within a prepared surgical opening in subchondral bone of a patient at an articular cartilage site, the surgical opening having a three-dimensional contour other than a circular cylinder, the implant comprising:
a plug body with a stabilizing portion for receipt within the surgical opening;
said stabilizing portion of said plug body presenting a three-dimensional contour other than a circular cylinder and configured for mated receipt within the surgical opening to provide a mechanical interlock against rotation.

49. A method for repairing articular cartilage in a patient, comprising:
implanting a first osteochondral plug graft at an articular cartilage site in the patient, the first osteochondral graft having a first body of bone and a first cartilage layer attached to the first body of bone;
implanting a second osteochondral plug graft adjacent to the first osteochondral plug graft at the articular cartilage site, the second osteochondral graft having a second body of bone and a second cartilage layer attached to the second body of bone; and
wherein first and second bodies of bone, as implanted, are in a nested relationship with one another.

50. The method of claim 49, wherein said nested relationship further provides a mechanical lock against lateral separation of the first and second osteochondral plug grafts.

51. (canceled)

52. (canceled)

53. (canceled)

54. (canceled)

55. (canceled)

56. The method of claim 49, wherein said nesting relationship provides a mechanical interlock that provides resistance to rotation of at least one of said first graft and said second graft.

57. (canceled)

58. The method of claim 49 wherein the patient is human and the osteochondral plug graft is an allograft.

59. (canceled)

60. (canceled)

61. The method of claim 49, also comprising administering an osteogenic protein to the articular cartilage site.

62. The method of claim 61, wherein the osteogenic protein comprises a bone morphogenic protein (BMP).

63. The method of claim 62, wherein the BMP comprises recombinant human BMP-2.

64. (canceled)

65. A method for repairing articular cartilage in a patient, comprising:
implanting a first plug body at an articular cartilage site in the patient;
implanting a second plug body adjacent to the first plug body at the articular cartilage site; and
wherein first and second plug bodies, as implanted, are in a nested relationship with one another.

66. An implant for receipt within an opening in subchondral bone at an articular cartilage site of a patient, the implant comprising:
a plug body configured for receipt within said opening in subchondral bone, wherein said plug body has at least a portion having sidewalls presenting a cross sectional profile selected from
a) a non-circular profile that includes at least one circular arc;
b) a polygonal profile;
c) an ovate profile; and
d) a multi-lobed profile having two to four lobes.

67. The implant of claim 66, wherein the implant is an osteochondral graft including a bone plug having an upper surface, sidewalls depending from said upper surface, and a lower surface, and a layer of cartilage attached to the upper surface of the bone plug, and wherein said sidewalls present said cross sectional profile.

68. The implant of claim 67, wherein said cross sectional profile is a non-circular profile that includes at least one circular arc.

69. The implant of claim 68, wherein said cross sectional profile is defined by multiple intersecting circular arcs.

70. The implant of claim 66, wherein said cross sectional profile is a polygonal profile.

71. (canceled)

72. (canceled)

73. (canceled)

74. (canceled)

75. (canceled)

76. (canceled)

77. The implant of claim 66, wherein said cross sectional profile is multi-lobed having two to four lobes.

78. The implant of claim 77, wherein said lobes each include a circular arc.

79. An implant system for receipt within an opening in subchondral bone at an articular cartilage site in patient, comprising a first plug body and a second plug body, wherein said first and second plug bodies are configured to cooperate with one another to nest, to mechanically lock at least one of the bodies against rotation, and/or to mechanically lock the bodies against lateral separation, when implanted in the opening.

80. The implant system of claim 79, wherein the first and second plug bodies are osteochondral plug grafts.

81-93. (canceled)

94. A method for repairing an articular cartilage site in a patient, comprising:
providing a prepared surgical opening in subchondral bone of a patient at an articular cartilage site; and
inserting a plurality of plug bodies into said surgical opening, said plurality of plug bodies together providing a plug assembly substantially filling said opening.

95. The method of claim 94, wherein said plug assembly has from two to four plug bodies.

96. The method of claim 94, wherein said plug assembly provides an interference fit in said opening.

97. The method of claim 95, wherein said plug bodies are osteochondral grafts.

98. The method of claim 94, wherein said plug grafts each include a body comprised of a synthetic polymer.

99. An implant system configured for stable implantation within a prepared surgical opening in subchondral bone of a patient at an articular cartilage site, comprising a plurality of plug bodies together providing a plug assembly configured to substantially fill the surgical opening.

100. The implant system of claim 99, wherein said plug bodies have graft wall portions configured to mate with one another.

101. The implant system of claim 100, wherein said plug body wall portions are substantially straight walls.

102. The implant system of claim 101, wherein said plug bodies have rectangular cross-sectional profiles.

103. The implant system of claim 99, wherein the plug bodies are osteochondral plug grafts.
